(19) United States
(12) Patent Application Publication
(10) Pub. No.: US 2009/0311238 A1
(43) Pub. Date: Dec. 17, 2009

(54) TOPICAL COMPOSITION AND TREATMENT METHOD FOR CARTILAGE DAMAGE

(76) Inventor: Wilson T. Crandall, Mount Dora, FL (US)

Correspondence Address: WANG & HO, 66 HILLTOP ROAD, MILLINGTON, NJ, 07946, US

(21) Appl. No.: 12/482,611

(22) Filed: Jun. 11, 2009

Related U.S. Application Data
(60) Provisional application No. 61/060,485, filed on Jun. 11, 2008.

(51) Int. Cl. A61K 38/48

(52) U.S. Cl. 424/94.63

(57) ABSTRACT

A method of treating cartilage damage by topically administering an effective amount of a pharmaceutical composition comprising (a) a substance P inhibitor and (b)a proteolytic enzyme with an suitable pharmaceutical carrier. The pharmaceutical composition is capable of promoting cartilage regeneration in animal and human subjects.

### CROSS REFERENCE TO RELATED APPLICATION

This application claims benefit of U.S. provisional patent No. 61/060,485 filed Jun. 11, 2008, the contents of which are incorporated herein in its entirely by reference.

### FIELD OF THE INVENTION

This invention is related to pharmaceutical composition and method for treating diseases caused by cartilage damage. Particularly, it is related to topical composition comprising a substance P inhibitor and a proteolytic enzyme and treatment method for cartilage damage by promoting cartilage regeneration or regrowth in damaged locations using said topical composition. The composition may be preferably formulated as pluronic lecithin organo gel or a liposomal.

### BACKGROUND OF THE INVENTION

Mammalian subjects, including human beings, may sometimes suffer cartilage damage. The body contains three different types of cartilage: articular, fibrocartilage, and elastic cartilage, which are around joint surfaces, in the knee meniscus and vertebral disk, and in the outer ear, respectively. Cartilage is a complex, living tissue that lines the bony surface of joints. Its main physiological function is to provide shock absorption and enable the joints to withstand weight bearing through the range of motion needed to perform daily activities as well as athletic endeavors. Cartilage damage may be suffered in the articular cartilage of the femoral cond,yles and/or head, meniscus, the tibia plateau, patella, glenoid labrum, humeral head as well as other cartilaginous joints and in the soft tissues including the cruciate ligaments and patella as well as the joint capsule of the humeral head or hip. Such damage may be in the form of cartilage loss and/or tearing of the soft tissue components due to inflammation or lack of collagen and proteoglycan production. Articular cartilage damage is the most common type of cartilage damage, and can occur as a result either of injury or degeneration caused by wear and tear, for example, under conditions such as trauma, osteonecrosis, osteochondritis, etc. Depending on the extent of the damage, and the location of the injury, articular cartilage cells may heal. However, articular cartilage has no direct blood supply, so it has little or no capacity to repair itself.

A standard practice of modern medicine is to prescribe anti-inflammatory medications. However, it has become known that such treatment, in the long run, do more damage than good. It has been realized that both cortisone shots and anti-inflammatory drugs have been shown to produce short-term pain benefit, but both result in long-term loss of function and even more chronic pain by actually inhibiting the healing process of soft tissues and accelerating cartilage degeneration. In other words, it is known that the anti-inflammatory treatment approach does not repair the damaged cartilage. Furthermore, other side effects may result from long-term use of anti-inflammatory drugs, such as other sources of chronic pain, allergies, G.I. bleeding and renal dysfunction.

To repair the damaged cartilage, the conventional surgical methods have been used: (a) microfracture, a procedure by which the bone's outer layer is penetrated, exposing the inner layers whereby bone marrow cells therein can access the damaged area and fill in the gap of cartilage; (b) cartilage transfer, a procedure by which small plugs of cartilage from other parts of the joint is moved to damaged areas; and (c) cartilage implantation, also known as autologous chondrocyte implantation (ACI), a procedure by which some cartilage cells are taken out and grown in an artificial environment in a laboratory and then, sufficient amount of the cartilage cells are re-implanted into the damaged area. Those surgical procedures have disadvantages. For microfacture, the new cartilage filled in the gaps by the exposed bone marrow cells is not the same as normal joint cartilage, and may not hold up over time, and require post-operative rehabilitation. Both cartilage transfer and cartilage implantation procedures are largely limited to the knee joint with only small sized cartilage defect, not widespread arthritis. Cartilage implantation usually requires multiple surgeries and a long rehabilitation period, which can be more than a year. Clearly, there is need for new ways of cartilage regeneration.

The present invention discloses such a new way of regenerating cartilage, based on an idea of topically applying a substance P inhibitor in combination with a proteolytic enzyme. This idea indeed came as a surprise because this composition was known for its anti-inflammatory effect and it was also known that the anti-inflammatory “treatment approach does nothing to repair the deteriorated cartilage and, thus, does not alleviate the chronic pain that people with this condition experience.”

### SUMMARY OF THE INVENTION

An object of the present invention is to provide a topically applied pharmaceutical composition that can promote regeneration of cartilage in the damaged areas. The composition comprises a substance P inhibitor and a proteolytic enzyme. A preferred a substance P inhibitor is capsaicin and a preferred proteolytic enzyme is bromelain. This composition is suitable for long-term applications.

In accordance with another embodiment of the present invention, the composition comprises capsaicin and bromelain, wherein the capsaicin and bromelain are combined to form a composition that is pharmaceutically effective in promoting cartilage regeneration. The composition may be preferably formulated as pluronic lecithin organo gel or a liposomal.

In accordance with another embodiment of the present invention, the forgoing composition further comprises a of a 1% theophylline, which is believed to be able to produce additional cyclic AMP to synergize with the bromelain.

The various features of novelty which characterize the invention are pointed out with particularity in the claims annexed to and forming a part of the disclosure. For a better understanding of the invention, its operating advantages, and specific objects attained by its use, reference should be made to the descriptive matter in which there are illustrated and to described preferred embodiments of the invention.

### DETAILED DESCRIPTION OF THE INVENTION

U.S. Pat. No. 5,560,910 disclosed the combined use of substance P inhibitors and proteolytic enzymes as an anti-inflammatory composition for relief of pain or pruritus due to inflammation.

The capsaicin was the naturally occurring capsaicin oleo resin (pcca, houston, Tex.). The caspaicin is dissolved in 0.5 ml-0.75 ml of ethanol and then added to the 10 grams of soy lecitihin 10 ml of isopropyl palmitate with homogenization. The 20 g of bromelain is first triturated and then dissolved in 40 ml of distilled water which is then homogenized. 10 g of the mixture of bromelain and water is then added to the capsaicin mixture of soylecithin and isopropyl palmitate and homogenization is repeated.

16 g of the pluronic 127 (basf. Parsippany, N.J.) is dissolved in 80 ml of water and refrigerated for 24-36 hours with occasional stirring. 94 ml of the 16.5% pluronic mixture.

The pluronic lecithin organogel is obtained by adding 80 ml of the 16.5 pluronic mixture to the mixture of isopropyl palmitate, soylecithin, bromelain and capsaicin to make a 100 g formulation. Of course, other concentrations of pluronic, deemed suitable by people with ordinary skill in the art, may used to obtain a satisfactory result under a particular circumstance.

As disclosed in U.S. Pat. No. 5,560,910, bromelain is a protease composition that may be isolated from pineapple. The enzyme has been reported to have anti-inflammatory activity when administered orally or parenterally (see Taussig, S. J., “The mechanism of the physiological action of Bromelain”, Medical Hypotheses, 6; 99-104, 1980). Commercially available bromelain used in the manufacture of pharmaceuticals is not a chemically homogeneous substance, but its principal component is a proteolytic enzyme, which is a glycoprotein with a molecular weight of approximately 33,000 Daltons.

Capsaicin is an oleoresin that may be obtained by extracting cayenne pepper with ether. The synthetic capsaicin is trans-8-methyl-vanillyl-6-nonenamide. Capsaicin gels have been described in U.S. Pat. No. 5,178,879 as being effective in treating topical pain. In addition, capsaicin is available commercially in over-the-counter compositions intended for pain relief including lotions such as HEET, OMEGA OIL, SLOAN'S LINIMENT and ZOSTRIX.

Bromelain has been reported to be an anti-inflammatory agent, an inhibitor of platelet aggregation, an agent that increases proteolytic and fibrinolytic activity in blood, and as a selective prostaglandin inhibitor. Bromelain has been administered by injection and has been reported to be effective after oral administration. However, because bromelain is a macromolecule, it cannot be administered transdermally using prior art formulations.

With respect to the proteolytic enzyme component of the composition, it comprises, in one embodiment, bromelain extracted from the pineapple stem (Spectrum, Gardena, Calif.), a protease composition which has a proteolytic enzyme as a principle component. (Bromelain is the general name for a family of sulhydryl proteolytic enzymes.) Commercially available bromelain used in the manufacture of pharmaceuticals is not a chemically homogeneous substance, but the principal component is a proteolytic enzyme that is a glycoprotein. The molecular weight of bromelain is approximately 33,000 Daltons.

The capsaicin may be provided at a concentration of between 0.0001% and 2.5% by weight, with a preferred concentration of between about 0.001% to 0.0025% by weight and with the most preferred concentration of about 0.0015% by weight. (MK 869, hydro-cinnamoyl-(S-benzoyl-homocysteine)benzyl ester, drugs which antagonize the Substance P recepetor (NK 1 receptor), Compound A, CP-22721, NKP-608, 679769 NS, TAK-637, Proyl endopeptidase (PEP, EC), AA501, NeurokininA, Nepaduant aqnd SR48968 or other known to those skilled in the art)> The bromelain may be provided at a concentration of between 0.5% and 20% by weight, with a preferred concentration of between approximately 3.5% and 15% by weight, and with the most preferred concentration of approximately 5% by weight. It should be understood that other proteolytic enzymes can be used in the present invention, including, but not limited to, gelatinase and elastase and others known to those skilled in the art.

It should be noted that the quantity of the composition necessary for effective therapy will depend on many different factors, including the physiological state of the patient and other medicaments administered. Thus, treatment dosages should be titrated to optimize safety and efficacy. Typically, dosages used in vitro may provide useful guidance in the amounts useful for in situ administration of the active ingredients in a quantity sufficient to constitute a therapeutic dose. Animal testing of effective doses for treatment of particular disorders will provide further predictive indication of a human therapeutic dosage. Various considerations are described, for example, in Goodman and Gilman's The Pharmacological Basis of Therapeutics, 7th Edition (1985), MacMillan Publishing Company, New York, and Remington's Pharmaceutical Sciences 18th Edition, (1990) Mack Publishing Co, Easton, Pa. Furthermore, as a topically applied pharmaceutical composition, the concentration can be varied in a much wider range than with orally administered medicines and injections and can be readily adjusted according to specific situations by the practitioners. Thus, the concentrations provided in the above are for example only, but not presented as limitation to the present invention.

The composition of the present invention is preferably loaded into a liposome, for topical administration to a human or animal patient, such as Natipide II preformed liposomes (American Lecithin CO, Oxford). Similarly, the method of Mezzei, et al., U.S. Pat. No. 4,937,078, incorporated herein by reference in its entirety, may be utilized. Other formulations known to one skilled in the art-may also be used.

The method of the present invention involves topical administration of a pharmaceutically acceptable composition containing the Substance P inhibitor and/or the proteolytic enzyme, loaded into a liposome, at the location of the pain or discomfort experienced by the patient. Application may be for any desired period for which benefit is provided, whether in the form of pain/discomfort relief or cartilage re-growth.

The method of the present invention involves topical administration of a pharmaceutically acceptable composition containing a Substance P inhibitor and a proteolytic enzyme, preferably loaded into liposomes, at the location of the cartilage damage. Application may be for any desired period for which benefit is provided. This composition is suitable for relatively long term use, for example, it may be used indefinitely unless irritation occurred.

While topical delivery by liposomes has been described as a preferred a method, it is to be appreciated that other methods of topical administration may also be used within the confines of the invention. For example, as shown in Examples 1 and 2 below, the active ingredients may be delivered by other known methods.

In another embodiment, the composition is created as follows: 5.0 g of 1200 or 2400 G.D.U. bromelain (Spec;trum La, Gardena, Calif.) is triturated and added to 15-25 g of distilled water is added with magnetic stirring, followed by homogenization. 0.15 g of capsaicin is dissolved in 0.5-0.75 ml of 90% ethyl alcohol with magnetic stirring. The bromelain is then added to this beaker and homogenization is repeated for an adequate amount of time to evenly disperse the Substance P inhibitor. A preservative such as sorbic acid, which is optional, is added to the composition. Optional stablilizers, antioxidants, preservatives, humectants, regreasing agents, solvents or auxiliaries is added to complete the 100 g total weight and improve the stability and/or adhesiveness of the formulations. This formulation is then loaded into Naptide II liposomes (American Lecithin Co., Oxford, Conn.).

This composition is applied topically in a uniform manner to the injured joint once cutaneous healing has occurred. Preoperative x-rays are used to assess the amount of osteoarthritis and assess the amount of joint cartilage present in chronic injuries. Physical exam of an injured knee can determine the stability of the cruciate ligaments and if there are torn menisci. Athroscopic exam or MRI will be definitive for cartilage damage or the necessity of surgically repairing a partially torn cruciate ligament.

### EXAMPLE 1

An individual had arthroscopic surgery for a bilateral meniscus tear. While the pain diminished, ibuprofen was still occasionally necessary. The pain developed further in the left leg and reexamination demonstrated that chondro malacia of the left patella existed. A retinacular release was performed to reposition the patella and avoid contact with the area of chondro malacia. There was short-term improvement, but the pain returned in the left and right legs after six months. Ibuprofen was used at the maximum dosage. A 5% by weight bromelain and 0.0015% capsaicin composition was applied topically in a Pluronic lecithin organogel (PLO) b.i.d. The composition was used for a three month treatment period, during which time pain in the patella could not be evoked while climbing stairs or elicited by medial & lateral displacement. Chondromalacia patellae means “softening of the articular cartilage of the knee-cap.” The articular cartilage is the cartilage lining under the knee-cap that articulates with the knee joint. Under normal circumstances, it is smooth and shiny, so that it glides smoothly along the articular groove of the femur as the knee bends. When it “softens”, it may break down, causing irregularities along the undersurface of the patella. The etiology is variable and the problem is common even in the young. The shiny white cartilage surface of the patella is at the back of the patella, where it makes contact with the cartilage of the femur as the knee is bent and straightened. Damage to the patellar cartilage is often mirrored by damage to the femoral cartilage as well, and then this is referred to as ‘patello-femoral’ damage. Cartilage (joint surface) damage is serious as cartilage heals poorly, and damage may be the beginning of a process leading to arthritis.

### EXAMPLE 2

A three year old Labrador Retriever was examined for chronic lameness and an “anterior drawer sign” was discovered indicating a torn anterior cruciate ligament (ACL). X-rays did not reveal osteoarthritis. Surgery was done and a partial tear in the anterior horn of the lateral meniscus was resected. The color of the torn meniscus was “yellowish” from the normal pearly white. The ACL was debrided and 60 lb. monofilament was used to recreate the ACL. The patella was examined and found normal. There was limited area of cartilage loss on the distal femoral condyles. The animal went home with a 5% by weight bromelain and 0.0015% capsaicin composition to be used b.i.d. for three months. Suture removal was done at seven days and the dog was beginning weight bearing at that time. Exercise was limited to walking on the leash for one month and the dog was then reexamined. The dog was seen the next year or rountine vaccinations and lab work. The owner reported that the dog tolerated exercise very well. Shortly thereafter, the, dog was killed in a car accident and permission to do an autopsy was obtained. The previously injured knee was inspected upon autopsy. There was normal appearing cartilage on the distal femur and the meniscus was a pearly white. The patella was also normal in appearance.

For pain relief applications, the composition may be applied for a period of hours, with treatments repeated for a period of weeks or months, depending on the results produced. Treatment may be administered in advance of certain pain-causing activities, such as piano playing, typing, exercise, etc.

While there have been described and pointed out fundamental novel features of the invention as applied to a preferred embodiment thereof, it will be understood that various omissions and substitutions and changes in the form and details of the embodiment illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention.

I claim:

1. A method of treating cartilage damage by promoting cartilage regeneration by topically administering an effective amount of a pharmaceutical composition comprising (a) a substance P inhibitor and (b) a proteolytic enzyme with an suitable pharmaceutical carrier
